(19) United States
(12) Patent Application Publication
(10) Pub. No.: US 2021/0311044 A1
(43) Pub. Date: Oct. 7, 2021

(54) MULTI-ASSAY RAPID DIAGNOSTIC PANEL

(71) Applicant: INTELLIGENT MATERIAL SOLUTIONS, INC., Princeton, NJ (US)

(72) Inventors: Joshua E. Collins, Wallingford, PA (US); Howard Y. Bell, Princeton, NJ (US)

(73) Assignee: INTELLIGENT MATERIAL SOLUTIONS, INC., Princeton, NJ (US)

(21) Appl. No.: 17/221,140

(22) Filed: Apr. 2, 2021

Related U.S. Application Data
(60) Provisional application No. 63/004,221, filed on Apr. 2, 2020.

(51) Int. Cl. G01N 33/543; G01N 33/569

(52) U.S. Cl. CPC G01N 33/54386; G01N 2333/165; G01N 2333/11; G01N 33/56983

(57) ABSTRACT

A rapid diagnostic test strip, system, and methodology are disclosed. The test strips utilize a sample pad on a proximal portion of a substrate. When a sample (potentially containing various analytes) is provided to the sample pad, it is transported to a conjugate release pad located distally from the sample pad. The conjugate release pad includes two or more targeted materials, where each targeted material includes an upconverting rare-earth particle capable of conjugating to an analyte. The conjugated analytes are then transported distally along the test strip, where they may bind to one or more test lines. An absorbent pad is located distally from the test lines. The one or more test lines can the be briefly illuminated with one or more specific wavelengths of light that the rare-earth particles absorb, and the rare-earth particles then emit a response that can be detected and measured.

### CROSS-REFERENCE TO RELATE APPLICATIONS

The present application claims priority to U.S. Provisional Pat. App. No. 63/004,221, filed on Apr. 2, 2020, which is incorporated herein in its entirety.

### FIELD OF THE INVENTION

The present disclosure is drawn to lateral flow assay (LFA) diagnostic assay panels, and specifically panels designed to aide in the diagnosis and treatment of individuals potentially infected with one or more conditions, such as influenza, coronavirus, etc., and strain specific identification of immune response.

### BACKGROUND

In recent years, there has been a demand for more point-of-care diagnostic assays, including LFAs. LFAs can be used to test for various illnesses, and are compatible with a variety of sample specimens including saliva, fingerprick blood, serum, and nasopharyngeal swab.

The basic principle behind the LFA is simple: a liquid sample containing the analyte of interest moves, via capillary action, through various zones of a test strip, on which molecules that can interact with the analyte are attached. A typical lateral flow test strip will include overlapping membranes, possibly mounted on a stiffer backing. The sample is applied at one end of the strip (on a “sample pad”), which may include surfactants, buffer salts, etc., to ensure the analyte present in the sample will be capable of binding to the capture reagents of conjugates and on the membrane. The treated sample migrates through a “conjugate release pad,” where the sample contacts antibodies that are specific to the target analyte and are conjugated to a marker of some type—typically coloured or fluorescent particles such as colloidal gold or latex microspheres. The sample, together with the conjugated antibody bound to the target analyte, continue to move down the strip to a “detection zone”, which is a porous membrane (such as nitrocellulose) containing antibodies and/or antigens in separate lines, which react with the analyte bound to the conjugated antibody. Typically, there is a “test line” (for a positive result) and a “control line” (for proof that the test is working). The lines in many LFAs can be read by eye, although in some cases, a dedicated reader is utilized.

However, to date, no rapid discovery panel is available that uses rare-earth particles to aid in the detection of multiple illnesses, especially with only a single test line, without an on-strip calibration area. Further, no test strip allows for the inclusion of additional rare-earth particles to be placed on the test strip in order to pass information relevant to the test (such as what analytes are being tested, when the strip was manufactured, or what the expiry date might be). Further, to date no rapid discover panel is available that is able to provide species specific identification of coronaviruses and the elicited host immune response.

### BRIEF SUMMARY

A first aspect of the present disclosure is drawn to a rapid diagnostic test strip, where a sample is applied near one end of the test strip, on a sample pad, and the sample generally flows in a direction towards an opposite end of the test strip. The test strip generally comprises at least five components: (i) a substrate; (ii) a sample pad on a proximal portion of the substrate, adapted to receive a sample; (iii) a conjugate release pad located distally from the sample pad; (iv) at least one test line (which optionally may be oriented perpendicular or angled to the direction of flow) located distally from the conjugate pad, the test line being adapted to bind to at least one analyte in the sample; and (v) an absorbent pad located distally from the at least one test line and the conjugate release pad. The conjugate release pad contains at least two conjugating materials, where each conjugating material is adapted to conjugate to an analyte, and each conjugating material comprising a rare earth particle. Each rare earth particle has a single pure crystalline phase of a rare earth-containing lattice, a uniform three-dimensional size, and a uniform polyhedral morphology.

In some embodiments, each analyte is one or more of coronavirus antigen variants, an influenza antigen, IgM, IgG, IgA, or one or more cytokines.

In some preferred embodiments, at least one test line of a given strip is configured to bind to two or more analytes.

In some preferred embodiments, the conjugate release pad also contains a control material, comprising at least one rare-earth particle that is different from other rare-earth particles used for the two or more conjugating materials. In preferred embodiments, the test strip further comprises a control line separately located from the at least one test line, between the conjugate release pad and the absorbent pad.

In some embodiments, at least one of the two or more conjugating materials is configured to absorb at least one different wavelength from at least one other rare-earth particle in the conjugate pad. Alternatively, or in addition to absorbing at different wavelengths, in some preferred embodiments, at least one of the two or more conjugating materials is configured to emit at least one different wavelength from at least one other rare-earth particle in the conjugate pad.

In preferred embodiments, the test strip further comprises a diagnostic pad, positioned between the conjugate pad and the absorbent pad, and any test lines and control lines are located on a top surface of the diagnostic pad.

In some embodiments, the test strip further contains rare-earth particles in a position separated from the test lines, the rare-earth particles selected to encode information about the test strip. In particular, these particles may be on the top surface of a diagnostic pad, and one or more variables (such as a temporal variable) relating to the emission profile of the rare-earth particles in this position are used to identify types and/or locations of the phosphors, and the locations/types of phosphors encodes the information about the test strip (such as which analytes the test strip is configured to assay, a date, or a combination thereof).

A second aspect of the present invention is drawn to a rapid diagnostic system, using the test strips. In particular, the system comprises at least one of the disclosed rapid diagnostic test strips and a reader adapted to detect the presence of a rare-earth particle bound to a test line. A remote server or database may optionally be utilized.

Preferably, the reader contains a processor configured to “read” the rare-earth particles in a particular fashion. In particular, the processor is preferably configured to cause the reader to emit at least one wavelength of light that a rare-earth particle on a test strip is capable of absorbing; allow the reader to detect an emission profile of the rare-earth particle in response to the at least one wavelength of light after at least one wavelength of light is no longer being emitted; determine if a parameter of the responsive emission profile is above a predetermined threshold; determine a temporal response of the rare-earth particle based on the detected emission profile; and providing a test result (e.g., by sending determined results to a display) based on the temporal response.

Preferably, the rapid diagnostic system comprises a first test strip configured to assay for an influenza virus and an assay for a coronavirus. Optionally, the first test strip is also configured to assay for IgM, IgG, IgA, at least one cytokine, or a combination thereof. Optionally, the test strips of the system comprise: a singleplex Multi-Antibody Assay (IgM, IgG, IgA); a multiplex Antibody Assay (IgM, IgG, IgA); a singleplex Cytokine Assay (Cytokine Panel); a multiplex Cytokine Assay (Cytokine Panel); a multiplex Antibody/Cytokine Assay (IgM, IgG, IgA/Cytokine Panel); or a combination thereof.

A third aspect of the present disclosure is drawn to a method for rapidly and securely diagnosing an infection. The method comprises first applying at least a portion of a test sample from a patient to a sample pad on a disclosed rapid diagnostic test strip, and allowing the sample to flow across the rapid diagnostic test strip for a predetermined period of time (such as between 1 minute and 5 minutes). Then, illuminating rare-earth particles on at least one test line with at least one wavelength of light the rare-earth particles are responsive to. In preferred embodiments, the at least one test line will have two or more different conjugated materials bound to it, so illuminating at least one test line will involve illuminating two or more different rare-earth particles, each rare-earth particle conjugated to a different analyte.

The illuminated rare-earth particles will then respond by emitting light. That response is measured. A determination is then made as to whether the first response is above a predetermined threshold.

In preferred embodiments, the response may be compared to a value in a database to determine what analyte is being assayed.

In some embodiments, at least one rare-earth particle attached to the test strip outside of the at least one test line is also illuminated. In such embodiments, a response from these rare-earth particles is also measured. That response is compared to a database to determine information about the test strip, the validity of the assay is determined based on that determined information, and the results of the assay are displayed.

### BRIEF DESCRIPTION OF THE DRAWINGS

FIG. 1A is an example of a test strip.

FIG. 1B is an example of a test strip in a cartridge.

FIGS. 2A and 2B are examples of alternative test strips.

FIG. 2C is a block diagram of an embodiment of a system using a test strip.

FIG. 3 is a graph illustrating a typical IgM/IgG antibody cycle during course of an infection and re-infection. Ratios of IgM/IgG can be accurately measured to aide in determining stage of infection.

FIG. 4 is a graph showing results from an analysis of 100 serum samples for HIV from 49 sero-negatives and 51 sero-positives. The highest negative value was 0.061.

FIG. 5A is graph illustrating the analytical sensitivity of a disclosed system vs. ELISA for an assay comparison for Schistosoma Detection (CAA).

FIG. 5B is a graph illustrating the limits of detection (LOD) of a disclosed system for CAA detection.

FIG. 5C is a comparison of various assay formats for detection of digoxgenin from V. chlorae.

FIG. 6A is a graph showing Ratio signal of test line signal (intensity) to control lines signal (intensity) intensities for 5 different analytes for leprosy detection.

FIGS. 6B-6D are examples of screenshots from a system for Positive, Borderline positive and Negative clinical samples.

FIG. 7A is a depiction of a Multiplexed Slanted Linear Array test strip for various cytokines.

FIG. 7B is a screenshot of a multiplexed LF array results for the diagnosis of active TB infection.

FIG. 8 is a flowchart of a proposed testing methodology and use case for a COVID-19 Rapid Diagnostic Kit during an active pandemic.

FIG. 9 is a general flowchart of a method for using the test strips to rapidly and securely diagnose an infection.

### DETAILED DESCRIPTION

The presently disclosed test strips, system, and methods involve lateral flow (LF) test strips and/or cartridges containing capture antibody and/or antigen and rare-earth particle-based conjugates which are analyzed using a point-of-care diagnostic reader obtaining results within 5 minutes of sample administration to the LF cartridge. In some embodiments, the test strips include antigen tests for other common pathogens, such as influenza, that can be referred to in case of a negative result on, e.g., a COVID-19 antigen test.

When provided as a kit, the LF diagnostic kit may include multiple LF assays intended to be used throughout the infection cycle from initial diagnosis to disease progression, treatment efficiency and recovery. The rapid diagnostic kit will include the appropriate sample collection devices and reagents for compatibility with a variety of sample specimens such as saliva, fingerstick blood, nasopharyngeal swab, plasma and serum. The kit may consist of a combination of multiplex and singleplex assays depending on the intervention stage and sample specimen.

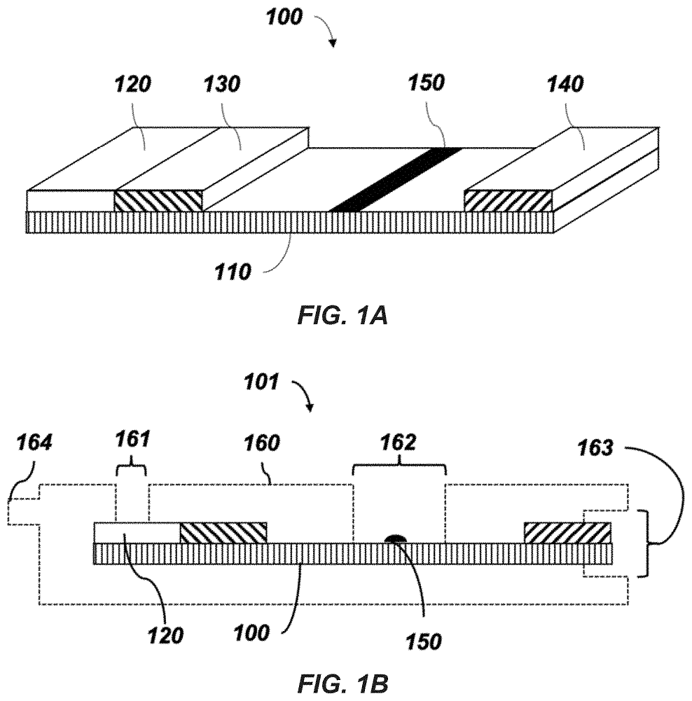

A general description of the disclosed rapid diagnostic test strips can be described with reference to FIGS. 1A and 1B. In FIG. 1A, one embodiment of a test strip (100) will generally comprises a sample pad (120), a conjugate pad (130), an absorbent pad (140) and at least one test line (150) placed on or above a substrate (110).

In FIG. 1B, a cartridge (101) is shown, where the test strip (100) is positioned within a housing (160) the housing defining at least one opening (161, 162). The at least one opening is configured to allow a sample to be placed directly on the sample pad (120), and preferably also allow each test line (150) to be directly viewable. In some embodiments, the at least one opening (161, 162) is a single large opening over both the test line (150) and the sample pad (120). In other embodiments, the housing defines one opening (161) over the sample pad (120) and a view window or second opening (162) over a test line. In some cases, additional openings are defined, such as one over each test line, or one over another specific portion of the test strip.

In some embodiments, the test strip (100) can be removed from the housing (101) through a defined opening (163). In other embodiments, the housing does not contain a defined opening for removing the test strip.

The housing is preferably a polymeric material, although other rigid materials can be utilized as well.

In some embodiment, one or more portions (164) of the housing may extend from the remainder of the housing, the extension optionally being shaped to allow a robotic arm to grab and maneuver the cartridges, or to allow a human hand to comfortably grab and hold the cartridge.

The substrate (110) may be any appropriate substrate known to skilled artisans, including, e.g., hydrophilic substrates such as thin layer chromatography substrates, cloth, paper, glass fibers, and polymers.

The sample pad (120) is generally position at a proximate portion of the test strip (100). The sample pad (120) may be any appropriate wicking material, including, e.g., woven or nonwoven cellulosic or polymeric fibers. Typically, the sample pad will comprise cellulose fiber filters and/or woven meshes (e.g., glass fiber). Many sample pad substrates are commercially available.

The sample pad (120) will generally be configured to promote a uniform and controlled distribution of the sample on the conjugate pad (130). The configuration of the sample pad controls the rate at which liquid flows into the conjugate pad, thereby preventing the device from overflowing. Thus, the materials, pore sizes, etc., are typically controlled based on the desired flow characteristics.

Further, the sample pad may optionally function as a carrier for pretreatment of solutes, etc. In some embodiments, the sample pad (120) can be impregnated with one or more additives. Such additives may include, e.g., proteins, detergents, tackifiers, buffer salts, or retarding agents. In some cases, the additives act a as filter, to prevent unwanted particulates, etc., from flowing through the test strip.

The treatment of the sample pad with blocking reagents, protein, detergents, and surfactants is a generally known practice. Treating the sample pad with an optimized buffer can normalize a sample before it reaches the conjugate pad, preventing undesirable interactions that may occur from, e.g., differences in pH, protein composition, mucins, salt concentrations, or molecules that cause non-specific interactions with the antibody system. Treatment buffers can normalize the sample pH and salt concentration, act as blocking agent for any non-specific binding, improve flow, and enhance the reproducibility of the assay by incorporating proteins, surfactants, salts, and/or polymers at the appropriate concentrations. The treatments are generally specific to the type of sample expected. For example, to determine what to include in the sample pad treatment, evaluate what aspect of the sample needs to be “normalized.” For saliva samples, one challenge may be the difference in viscosity of the samples. Saliva samples may be treated with increased salt and surfactant concentrations to alter the viscosity of the samples, but such a treatment lead to undesirable effects with a whole blood sample (e.g., hemolysis of the red blood cells, transport of the lysed cells through the test strip, etc.)

Treatments can be applied using known techniques. For example, a treatment can be performed by immersion or spray deposition. Following treatment, the sample pad may optionally be cured (such as in a forced air convection oven at 35-40° C. for 60 minutes, and then dried overnight at room temperature in a <20% relative humidity environment. When sample pads are cured in this manner, they will preferably by stored and maintained in a dry environment (<20% relative humidity) at room temperature (18-25° C.) to avoid additional moisture uptake.

The conjugate pad (130) is, like the sample pad, generally a known wicking material, such as cellulose fiber filter or glass fiber. Many conjugate pad substrates are commercially available.

Generally, the conjugate pad is configured to provide a substantially uniform conjugate release. By controlling bed volume (the total volume of air in the conjugate release pad) via density and thickness of the conjugate release pad substrate, one can control critical in conjugate release. Generally, lower bed volume equates to faster release.

The conjugate pad substrate may be pretreated. In some embodiments, the pre-treatment include a salt buffer, proteins, polymers, and detergents that can aid in release of the conjugate from the conjugate pad. In some embodiments, components of pretreatment are selected to help block protein binding sites on the membrane prior to the conjugate interaction, as the conjugate pad treatment buffer will also move up the strip faster than the conjugate. This will lead to less non-specific binding, and higher sensitivity.

The conjugate pad can be treated and optionally dried, in the same fashion as the sample pad.

Any known techniques for applying the conjugates to the conjugate pad can be used. In some embodiments, the conjugates are applied to the conjugate pad with the use of an air jet dispensing platform or by immersion. Often, a buffer is used to apply the conjugate onto the conjugate pad.

After applying a conjugate onto the conjugate pads, the pads can be dried, using the same drying techniques as discussed previously for sample pads.

In some embodiments, the conjugated materials can be dried or lyophilized onto the conjugate release pad at a concentration range from 100 ng to 1.0 μg of conjugate.

Each conjugating material is generally configured to bind to conjugate to a specific analyte in the sample.

The test strip will comprise two or more conjugating materials configured to conjugate to different tested analytes. The tested analytes for a test strip will preferably comprise a coronavirus antigen, an influenza antigen, IgM, IgG, IgA, one or more cytokines, or a combination thereof. For example, in some embodiments, a test strip comprises a second conjugating material comprising a recombinant trimerized form of the S protein and/or the central portion of this molecule defined as the receptor binding domain (RBD), consisting of amino acid sequences specific for the COVID-19 variant B.1.1.7 attached to a first rare-earth particle, and a second conjugating material comprising a recombinant trimerized form of the S-protein and/or the RBD of this protein of a differing amino acid sequence specific to variant P.1, attached to a second rare-earth particle. The capture analytes are sprayed in single test lines consisting of anti-human IgM, anti-human IgG, and anti-human IgA antibodies. The control line will have antibodies specific to the different recombinant protein variants on the nanoparticle conjugates. The nanoparticle RBD conjugates for the different variants will be dried on the sample conjugate pad. In this embodiment, for example, a fingerstick blood specimen will be collected and mixed in an assay buffer. A specific volume, typically 50-100 ul of specimen in buffer will be applied to the sample pad on the lateral flow strip, reconstituting the dried conjugates and initiating binding of the target antibodies to the conjugates and the capture antibodies on the respective test lines. The lateral flow cartridge will be scanned using a laser-based detection device whereby each test line and control lines are interrogated and emission from each optical reporter collected and analyzed. The different nanoparticle conjugates will emit either unique spectral lines and/or possess unique time domain responses that can be accurately identified and quantified.

In some embodiments, the conjugate release pad further comprises a control material. This control material is configured to act as the signaling agent for control purposes. In such arrangements, it is configured to not conjugate an analyte or to conjugate to an analyte that is present in the sample, but is not one of the target analytes or an analyte that is otherwise relevant for the purposes of the test strip. That control material will, however, bind to a test line or control line distally located on the test strip. As it is being used as a signaling agent for control purposes, if a separate control material is present, it will preferably comprise at least one rare-earth particle that is distinct from the rare-earth particles present in the conjugating materials.

The conjugating materials that are applied will each comprise a rare-earth particle reporter, and preferably an upconverting rare-earth particle (although downconverting rare-earth particles could also be utilized). In preferred embodiments, each rare-earth particle is one of a plurality of monodisperse particles, the particles each having a single pure crystalline phase of a rare earth-containing lattice, a uniform three-dimensional size, and a uniform polyhedral morphology.

In preferred embodiments, each group of conjugating materials utilizes a unique rare-earth particle composition and/or morphology, that is a plurality of monodisperse particles, the particles in each group having a single pure crystalline phase of a rare earth-containing lattice, a uniform three-dimensional size, and a uniform polyhedral morphology. Each analyte is thereby associated with a unique group of rare-earth particles, that can be uniquely identified via its emission profile from the rare-earth particles associated with other analytes.

In some embodiments, at least one of the two or more conjugating materials is configured to absorb at least one different wavelength from at least one other rare-earth particle in the conjugate pad. For example, in some embodiments, the rare-earth particles may emit at substantially the same wavelength of light, but one conjugating material for a first analyte utilizes an upconverting rare-earth particle, and a second conjugating material for a different analyte uses a downconverting rare-earth particle, such that they will necessary absorb (or be activated by) different wavelengths of light.

In some embodiments, at least one of the two or more conjugating materials is configured to absorb light having at least one wavelength that is the same as at least one other rare-earth particle in the conjugate pad, and emit light having a peak wavelength that is different from the peak wavelength emitted by at least one other rare-earth particle in the conjugate pad. For example, in one embodiment, each conjugating material absorbs around 940 nm, but one emits a green light and one emits a red light.

Upconverting Phosphors (UCP) Reporters

A large variety of up-converting inorganic rare-earth particle compositions are also known in the art. As is known in the art, up-converting rare-earth particles derived from RE-containing host lattices, such as described above, doped with at least one activator couple comprising a sensitizer (also known as an absorber) and an emitter. Suitable up-converting rare-earth particle host lattices include: sodium yttrium fluoride (NaYF₄), lanthanum fluoride (LaF₃), lanthanum oxysulfide, RE oxysulfide (RE₂O2_{S}), RE oxyfluoride (RE₄O₃F₆), RE oxychloride (REOCl), yttrium fluoride (YF3), yttrium gallate, gadolinium fluoride (GdF₃), barium yttrium fluoride (BaYF₅, BaY₂F₈), and gadolinium oxysulfide, wherein the RE can be Y, Gd, La, or other lanthanide elements. Suitable activator couples are selected from: ytterbium/erbium, ytterbium/thulium, and ytterbium/holmium. Other activator couples suitable for up-conversion may also be used. By combination of RE-containing host lattices with just these three activator couples, at least three rare-earth particles with at least three different emission spectra (red, green, and blue visible light) are provided. Generally, the absorber is ytterbium and the emitting center can be selected from: erbium, holmium, terbium, and thulium; however, other up-converting rare-earth particle particles of the invention may contain other absorbers and/or emitters. The molar ratio of absorber:emitting center is typically at least about 1:1, more usually at least about 3:1 to 5:1, preferably at least about 8:1 to 10:1, more preferably at least about 11:1 to 20:1, and typically less than about 250:1, usually less than about 100:1, and more usually less than about 50:1 to 25:1, although various ratios may be selected by the practitioner on the basis of desired characteristics (e.g., chemical properties, manufacturing efficiency, excitation and emission wavelengths, quantum efficiency, or other considerations). For example, increasing the Yb concentration slightly alters the absorption properties, which is useful for biomedical applications. The rare-earth particle of the invention can be excited at 915 nm instead of 980 nm where the water absorption is much higher and more tissue heating occurs. The ratio(s) chosen will generally also depend upon the particular absorber-emitter couple(s) selected, and can be calculated from reference values in accordance with the desired characteristics. It is also possible to control over particle morphologies by drastically changing the ratio of the activators without the emission properties changing drastically for most of the ratios but quenching may occur at some point.

The optimum ratio of absorber (e.g., ytterbium) to the emitting center (e.g., erbium, thulium, or holmium) varies, depending upon the specific absorber/emitter couple. For example, the absorber:emitter ratio for Yb:Er couples is typically in the range of about 20:1 to about 100:1, whereas the absorber:emitter ratio for Yb:Tm and Yb:Ho couples is typically in the range of about 500:1 to about 2000:1. These different ratios are attributable to the different matching energy levels of the Er, Tm, or Ho with respect to the Yb level in the crystal. For most applications, up-converting rare-earth particles may conveniently comprise about 10-30% Yb and either: about 1-2% Er, about 0.1-0.05% Ho, or about 0.1-0.05% Tm, although other formulations may be employed.

Some embodiments of the invention employ inorganic rare-earth particles that are optimally excited by infrared radiation of about 950 to 1000 nm, preferably about 960 to 980 nm. For example, but not by limitation, a microcrystalline inorganic rare-earth particle of the formula YF₃:Yb_{0.10}Er_{0.01} exhibits a luminescence intensity maximum at an excitation wavelength of about 980 nm. Up-converting rare-earth particles of the invention typically have emission maxima that are in the visible range. For example, specific activator couples have characteristic emission spectra: ytterbium-erbium couples have emission maxima in the red or green portions of the visible spectrum, depending upon the rare-earth particle host; ytterbium-holmium couples generally emit maximally in the green portion, ytterbium-thulium typically have an emission maximum in the blue range, and ytterbium-terbium usually emit maximally in the green range. For example, Y_{0.80}Yb_{0.19}Er_{0.01}F₂ emits maximally in the green portion of the spectrum.

Particle Properties Based on Composition, Morphology, and Size

Properties of the monodisperse particles can be tuned in a variety of ways. As known in the art and discussed above, the properties of the monodisperse particles, the characteristic absorption and emission spectra, may be tuned by adjusting their composition, e.g., by selecting a host lattice, and/or by doping. Additionally and advantageously, given their uniform polyhedral morphology, the monodisperse particles of the invention exhibit anisotropic properties. Particles of the same composition but different shape exhibit different properties due to their shape and/or size. In one exemplary embodiment of the invention, the monodisperse particles, particularly UCNP's, of the invention are varied in composition and/or shape to give different decay lifetimes. Having different spectral decay lifetimes allows unique rare-earth particle to be differentiated from one another. The ability to have monodisperse particles of the same composition but different morphologies according to the invention permits use of one composition (especially in regulated industries such as pharmaceuticals or medical devices) but to distinguish its morphologies through their unique optical properties. However, to take advantage of this, to tune the particle and its optical properties in this way, has not been possible but is now achieved with the monodisperse particles of the invention.

Thus, in addition to the characteristic absorption and emission spectra that can be obtained the rise and decay times of a monodisperse particle of the invention can also be tuned by particle size and morphology. The rise time is measured from the moment the first excitation photon is absorbed to when the first emission photon is observed. The decay time is measured by the slope of the emission decay, or the time it takes for the rare-earth particle to stop emitting once the excitation source is turned off.

By changing the dopant ratio, the rise and decay times can be reliably altered.

Briefly, typically an excited state population decays exponentially after turning off the excitation pulse by first-order kinetics, following the decay law, I(t)=I0 exp (−t/τ), whereby for a single exponential decay I(t)=time dependent intensity, I0=the intensity at time 0 (or amplitude), and τ=the average time a rare-earth particle remains in the excited state (or <t>) and is equal to the lifetime. (The lifetime τ is the inverse of the total decay rate, τ=(T+knr)−1, where at time t following excitation, T is the emissive rate and knr is the non-radiative decay rate). In general, the inverse of the lifetime is the sum of the rates which depopulate the excited state. The luminescence lifetime can be simply determined from the slope of a plot of Inl(t) versus t (equal to 1/τ). It can also be the time needed for the intensity to decrease to 1/e of its original value (time 0). Thus, for any given known emission wavelength, a number of parameters fitting the exponential decay law can be monitored to identify a particular rare-earth particle or group of rare-earth particles, thus permitting their use, for example, in developing unique anti-counterfeiting codes, signatures, or labels/taggants.

In most instances, lifetimes are controlled by variations in the crystal composition or overall particle size. However, by controlling the particle morphology and uniformity as with the monodisperse particles of the invention one can create particles of visually distinct morphologies possessing lifetimes that are unique to that morphology while maintaining identical chemical compositions among the various morphologies. This feature allows for a highly complex optical signature or taggant which, as discussed above, may be used in serialization and multiplexing assays or analysis in various fields such as, for example, assays, biomedical, optical computing, as well as use in security and authentication.

To illustrate, consider the dependences of upconversion luminescence (UCL) on the particle size, shape, and inorganic-ligand interface of the hexagonal (β)-phase NaYF₄:Yb,Er upconverting nanorare-earth particles of the invention. The relative luminescent intensity, power-dependent luminescence, green to red emission intensity ratio (fg/r), and dynamic luminescence lifetimes of the prism-, plate-, and rod-shaped hexagonal (β)-phase NaYF₄:Yb,Er particles of the invention as a function of surface to volume (SA/Vol) ratio was measured. The upconverting properties of the particles can be attributed to not only the surface effects by comparing the SA/Vol ratios but also the particle morphologies or shapes. At the comparable SA/Vol and ion (Yb/Er) doping ratios (20%/2%), the prism-shaped nanocrystal particles showed increased intensity and smaller saturation power than those of the rod-shaped nanocrystals. Therefore, the differently shaped nanocrystals with identical SA/Vol ratios could have different lattice energy and multiphonon relaxation processes. Such rare-earth particles can be prepared as provided in, e.g., U.S. Pat. No. 9,181,477, incorporated by reference herein in its entirety.

The UCP reporter technology is a significant advancement over the current state of the art analysis techniques. It entails multi-photon infrared excitation and subsequent emission of higher energy visible light. The total absence of autofluorescence provides the label with a distinctive advantage compared to common fluorescent labels and extra sensitivity, enabling detection days before a test with other labels such as gold or fluorescent dyes. Additionally, samples require little to no preparation, can be administered by low-technology operators and provide results in as little as 5 minutes.

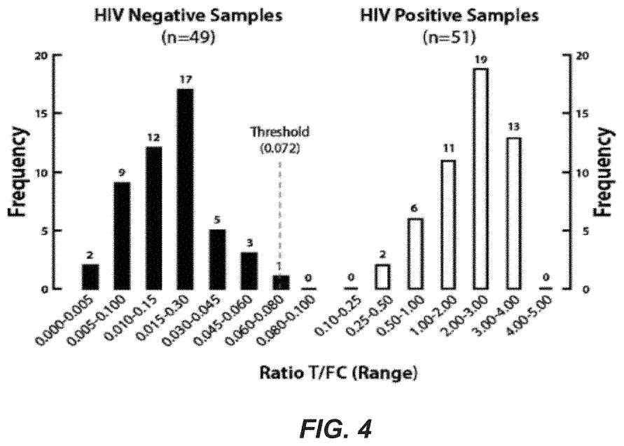

Depending on the selected UCPs, the disclosed platform enables highly resolved quantification resulting in greater separation between antibody positive and negative groups, thus better Sn/Sp. See, e.g., FIG. 4, which illustrates the use of the platform for discriminating HIV test results.

From the conjugating pad (120), the sample flows (via, e.g., capillary action) distally towards at least one test line (150). The test line will generally comprise appropriate proteins, either antibody or antigen as appropriate, that have been laid down to capture the target analyte and conjugate as they migrate up the strip.

In preferred embodiments, the at least one test line comprises or consists of a single line, where the single line is capable of binding to two or more analytes. Rather than having separate lines, a single line can be used to bind to multiple analytes. In preferred embodiments, the single lines have a plurality of binding sites that are each, independently, configured to specifically bind to a given analyte. For example, for a test line that tests for a influenza antigen and a coronavirus antigen, the single line could contain two different proteins, one for binding the influenza antigen and one for binding the coronavirus antigen.

In more preferred embodiments, every test line, including a control line, is capable of binding to two or more analytes.

In a most preferred embodiment, every test line, including a control line, is capable of binding to all analytes conjugated to a conjugating material. That is, in a most preferred embodiment, the test strip contains two lines—a test line and a control line—and each line captures each of the two or more conjugated materials.

In some preferred embodiments, some or all of the control line is located distally from the at least one test line. In other preferred embodiments, the control line is offset in a direction perpendicular to the direction the sample flows across the test strip (the “flow direction”), but is substantially the same distance from the sample pads in the flow direction.

In some embodiments, there is a single control line on a test strip. In other embodiments, there is a single control line for every test line on the test strip. In still other embodiments, there is a plurality of control lines, but less than the number of test lines.

In one example, a lateral flow test will be capable of detecting the presence of a COVID-19 antigen infection or influenza on a single test line containing antigen specific to COVID-19 and influenza as the capture molecules, as well as determine if disease progression through cytokine detection and quantification on a second test line using anti-IL-6, anti-IL-10 and anti-CRP antibodies as capture molecules. In this example there are two separate control lines where the first control line contains COVID-19 and influenza antigen as capture molecules and the second control line contains cytokines IL-6, IL-10 and CRP. In total 5 unique rare earth nanoparticle reporters will be utilized for the conjugate. The conjugate molecules will be as follows: COVID-19 nucleocapsid antibody, Influenza antibody, and IL-6, IL-10 and CRP antibodies. The five conjugates will be dried on the conjugate/sample pad on the lateral flow strip.

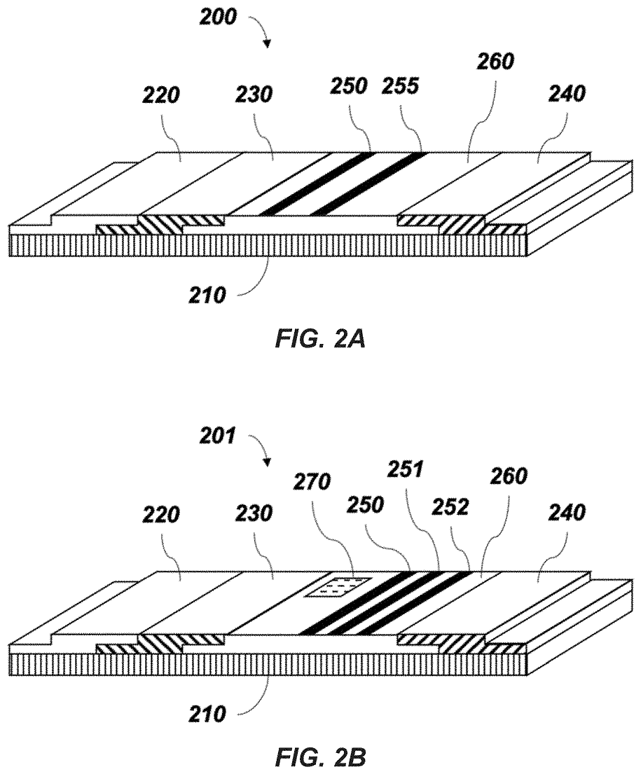

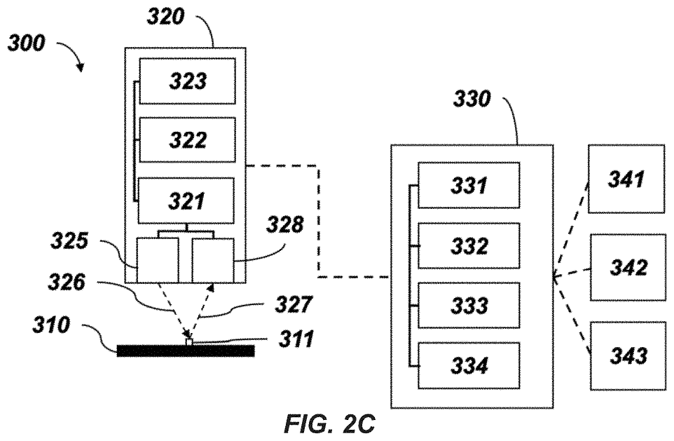

Referring to FIGS. 2A and 2B, in some embodiments of a test strip (200, 201), a backing substrate (210) is provided. A sample pad (220) is provided on the backing substrate. The sample pad at least partially overlaps the conjugate pad (230). The conjugate pad at least partially overlaps a diagnostic pad (260). The absorption pad (240) at least partially overlaps the diagnostic pad (260). The diagnostic pad is positioned between the conjugate pad and the absorbent pad.

The diagnostic pad substrate is another wicking substrate, and is typically comprised of a material such as a nitrocellulose membrane. In FIG. 2A, at least one test line (250, 251) and at least one control line (252, 255) are depicted on the top surface of the diagnostic pad (260). Here, the control and test lines are shown as being perpendicular to the flow direction. However, other orientations are possible. In some embodiments, the test and control lines are angled (that is, non-parallel and non-perpendicular) to the direction of flow.

As seen in FIG. 2B, in some embodiments of the test strip (201), at least one portion (270) of the diagnostic pad (260) may comprise one or more different rare-earth particles directly attached to the diagnostic pad, such as being attached directly to the top surface. The rare-earth particles in this portion are preferably one or more pluralities of monodisperse particles, the particles each having a single pure crystalline phase of a rare earth-containing lattice, a uniform three-dimensional size, and a uniform polyhedral morphology. The rare-earth particles may be the upconverting rare-earth particles as described above.

The rare-earth particles here are intended to provide information to a reader device about the test strip itself. The information is encoded, and is at least partially based on a variable related to the emission profiles of the rare-earth particles used here. For example, decay time can be used to identify the presence of distinct groups of rare-earth particles present in a given location. The information may include, e.g., which analytes the test strip is configured to assay, a date (such as an expiry date of the test or a date of manufacturing), or a lot number. In some embodiments, this information may optionally be encoded as a 1-D, 2-D, or 3-D barcode, and may be printed on the test strip.

The absorbent pad (140) is generally at a distal-most portion of the test strip. Generally speaking, the purpose of the absorbent pad is to increase the total volume of sample that can enter the test strip. The bed volume of any membrane is finite, and having an absorbent pad at the distal end of the test strip can increase the volume of sample that can be ran across the membrane as it acts as a sponge for the additional volume. As such, the presence of the absorbent pad contributes to the reduction of non-specific binding and sensitivity. This is accomplished due to the additional volume that can run across the test line washing non-specifically bound material off the test line, and allowing for an increase in total analyte concentration to reach the test line.

As seen in FIG. 2C, the test strip can be utilized as part of a disclosed system. In FIG. 2C, the system (300) utilizes a reader device (320) to “read” the test strip (310), and typically will communicate with a separate server, mobile device, or computer (330), which itself may be connected to various devices (341, 342, 343). In particular, the reader device (320) will typically contain at least a processor (321) and memory (322), and a wireless or wired communication interface (323). The memory will typically be a non-transitory computer readable storage media that contains instructions that, when executed, controls the processor on the reader and may cause the reader to perform certain activities at particular times as described below. The processor (321) is configured to control a source of radiation (325) (such as a laser or LED). That radiation directed towards (326) and absorbed by a rare-earth particle (311) on the test strip (310). The rare-earth particle (311) will then emit (327) radiation (which has an emission profile specific to that type of rare-earth particle), which is detected by a sensor (328) in the reader. The sensor then sends the detected signals to the processor (321). In some embodiments, the processor on the reader device transmits the signals as a payload in communication to a remove computer, mobile device, or computer (330). That remote computer (such as a remove server or smartphone) will generally contain a processor (331) and memory (332), and may optionally contain a separate database (333) of data related to the emission profiles received from the rare-earth particles on the test strips. The memory (332) will typically be a non-transitory computer readable storage media that contains instructions that, when executed, controls the processor on the remote computer or device (330) and may optionally control the performance of the reader (320), depending on the exact configuration desired. The computer or device (330) may also contain a wired or wireless communication interface (334) for communicating with remove devices, such as a separate display (341), a remote computer or mobile device (342), or another server or database (343).

A multiplexed cytokine assay panel can be utilized as, e.g., an initial triage screening to determine whether an infection is viral or bacterial based on the host-protein signature as well as a tool to monitor the progression in infected individuals. For initial screening, cytokine based assay panels can prove to be a reliable diagnostic tool for identifying bacterial vs. viral infections when antigen-based assays are unavailable such as in the early stages of the current COVID-19 pandemic. Utilizing antibody assays alone carries the risk of false negatives as individuals may be tested while symptomatic but if testing occurs too early in the infection the individuals may not yet have mounted a pathogen specific immune response which is why it is important to diagnose the presence of an infection using antigen or molecular based approaches.

Additionally, when the system is provided as a diagnostic kit, the kit may include singleplex antibody tests using a single test line containing anti-IgM, IgG, and IgA antibodies for a highly sensitive, user-friendly assay to be used to evaluate solely for previous exposure to a coronavirus such as COVID-19. Antibody detection tests are highly sensitive assays. The ratio between the various types of antibody (i.e. IgM vs IgG levels) can provide insight in the timecourse of infection. However, antibodies can only be detected in patients after sero-conversion which is after the viral peak concentrations when patients may already be symptomatic and contagious for several days. Antibody detection tests are useful in reassuring individuals that have gone through infection but not tested that they have had, e.g., a COVID-19 infection and are protected against re-infection.

This test can also be utilized to identify individuals who have been previously infected and presented little to no symptoms where specific IgM/IgG/IgA antibody ratios are not required. These tests can be extremely valuable in pandemic situations to identify possible donors whose plasma can be provided as a treatment to infected individuals. The typical detection ranges for each of the identified markers have been identified below in Table 1.

TABLE 1
Biomarker panel and analytical sensitivity
for a coronavirus Rapid Diagnostic Kit.

| Target Biomarker | Detection Range |
|---|---|
| CoV IgM | 5.0 pg to 100,000 ng/ml |
| CoV IgG | 2.0 pg to 100,000 ng/ml |
| CoV IgA | 10.0 pg to 100,000 ng/ml |
| CoV-Specific Antigen | 0.1 pg to 100 ng/ml |
| Cytokines | |
| IL-6 | 1 to 1,000 pg/ml |
| IP-10 | 100 to 100,000 pg/ml |
| CRP | 1 to 100,000 ng/ml |
| TNF-α | 10 to 10,000 pg/ml |

As stated above, in some embodiments, the assay formats utilized in a rapid diagnostic kit will be a combination of both singleplex and multiplex assays. A multiplexed cytokine assay employing a slanted linear array format (see PCT/US19/47432, which is incorporated by reference herein in its entirety) was demonstrated in a Tuberculosis rapid field diagnostic comprising of a panel of cytokines including IL-6, IP-10, CRP, and TNF-α. The rapid lateral flow diagnostic kits described in this invention can have various combinations of singleplex and multiplex assays comprising of, e.g., COVID-19 specific antigen and/or COVID-19 antibody and cytokine assays. The various formats for each of the assays in a diagnostic kit are described Tables 2 and 3, below. The disclosed test strips can be used for any of the assays where more than a single analyte is being assayed on a single strip.

TABLE 2
Example Assays.

| Type # | Description |
|---|---|
| 1 | Singleplex CoV Antigen Assays |
| 2 | Singleplex High Sensitivity, Multi-Antibody Test Line (IgM, IgG, IgA) |
| 3 | Singleplex Cytokine Assays (IL-6, IP-10, CRP, TNF-α) |
| 4 | Multiplex Antibody Assay (IgM, IgG, IgA) |
| 5 | Multiplex Cytokine Assay (Cytokine Panel) |
| 6 | Multiplex Antigen/Cytokine Assay (CoV-specific antibody + Cytokine Panel) |
| 7 | Multiplex Antibody/Cytokine Assay (IgM, IgG, IgA/IL-6, IP-10, CRP, TNF-α) |
| 8 | Multiplex Antigen/Antibody/Cytokine (IgM, IgG, IgA/IL-6, IP-10, CRP, TNF-α) |

TABLE 3
Descriptions of Example CoV Rapid Diagnostic Kits.

| # | List of Items In Kit |
|---|---|
| 1 | 1. Singleplex CoV Antigen Assays |
| | 2. Singleplex High Sensitivity, Multi-Antibody Test Line (IgM, IgG, IgA) |
| | 3. Singleplex Cytokine Assays (Cytokine Panel) |
| 2 | 1. Singleplex CoV Antigen Assays |
| | 2. Multiplex Antibody Assay (IgM, IgG, IgA) |
| | 3. Multiplex Cytokine Assay (Cytokine Panel) |
| 3 | 1. Singleplex CoV Antigen Assays |
| | 2. Multiplex Antibody/Cytokine Assay (IgM, IgG, IgA/Cytokine Panel) |
| 4 | 1. Singleplex CoV Antigen Assays |
| | 2. Multiplex Cytokine Assay (Cytokine Panel) |
| 5 | 1. Multiplex Antibody Assay (IgM, IgG, IgA) |
| | 2. Multiplex Cytokine Assay (Cytokine Panel) |
| 6 | 1. Multiplex Antigen/Antibody/Cytokine Assay (CoV specific antibody/IgM, IgG, IgA/ |
| | Cytokine Panel) |
| 7 | 1. Multiplex Antigen/Cytokine Assay (CoV specific antibody/Cytokine Panel) |

In the instance of a single line, multiplexed antibody assay the biomarkers used for the upconversion reporter conjugate and the test line capture biomarker will be recombinant proteins, S and/or RBD proteins, that have been produced by transfections using purified DNA from specific COVID-19 strains (i.e. P.1, B.1.1.7 or B.1.351) and possessing the mutations specific to those strains. The recombinant proteins utilized for the upconverting reporter conjugates will have each strain specific protein bound to an upconverting reporter with a unique optical property that can be easily differentiated either via spectral or time-domain analysis from other reporter conjugates.

The basic platform has been deployed worldwide as an effective tool for the detection of various infectious diseases, including Leprosy and Tuberculosis. These efforts have demonstrated the significant improvements in assay performance enabling orders of magnitude increase in sensitivity compared to standard gold and ELISA based platforms.

The unique sensitivity of the UCP based LF assays provide many benefits such as: (i) early detection due to increased sensitivity (100× more sensitive than gold); (ii) precisely controlled particle morphologies and optical properties enable highly resolved quantification to follow the disease progression; (iii) compatibility with a variety of sample specimens; saliva, finger-prick blood, serum, nasopharyngeal swab; (iv) rapid results: Leprosy UCP-LFA Test & Flow Control Ratios stabilized <5 min; (v) field deployable (e.g. “drive-through testing centers”): Low-resource/training operators can perform testing and provide results rapidly; (vii) Ease of Use: saliva & FPB specimens require no sample prep; and (viii) Cost Effective: The system can utilize standard LF cartridges and/or standard cartridge drawers if desired, and commercial off the shelf point-of-care readers and customized portable units based on lightweight reader optics and electronics.

The availability of this technology will have significant impact in the ability of clinicians and other healthcare providers to quickly, accurately, and quantitatively diagnose those infected with CoVs such as COVID-19. The capability of this platform to detect the earliest stages of infection will realize reduced transmission of the disease as well as the overall morbidity and mortality associated with more advanced stages. The technology developed has the added benefit to be utilized for numerous other biological targets and markers of disease that have global impacts on human health. The versatility of the disclosed rapid diagnostic platform will also significantly increase the ability to have a broad detection capability using a single reader system. In addition, there are several commercially available LF devices which are compatible with upconverting reporters.

The UCP reporter technology is a significant advancement over the current state of the art analysis techniques. It entails multi-photon infrared excitation and subsequent emission of higher energy visible light. The total absence of autofluorescence provides the label with a distinctive advantage compared to common fluorescent labels and extra sensitivity, enabling detection days before a test with other labels such as gold or fluorescent dyes. Additionally, samples require little to no preparation, can be administered by low-technology operators and provide results in as little as 5 minutes.

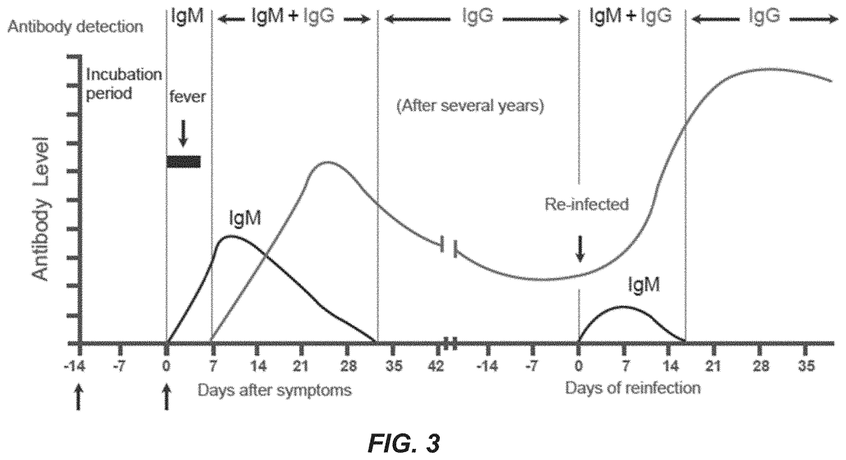

In some embodiments, detection of infection will focus on the IgM/IgG/IgA antibody response. FIG. 3 shows a typical cycle of IgM/IgG antibodies present during viral infections.

In this example, IgM, IgG, and IgA antibodies will be detected using a single test line and single control line strip. The capture molecules on the single test and control lines will be recombinant protein for the spike protein of COVID-19. The conjugates will use different nanoparticles with unique, differentiable optical properties bound to either anti-human IgM, IgG, or IgA which will bind any IgM, IgG, or IgA antibodies in the clinical sample. In practice the sample will be added to the conjugate release pad, mixing the nanoparticle conjugates with the sample allowing for binding of the conjugate to the target antibody. Subsequently the sample mixture flows across the LF membrane eliciting further antibody-antigen binding at the test and flow control lines. After the assay is complete the test and flow control lines will be scanned, looking for any of the three unique optical emissions from the different nanoparticle conjugates on the test and flow control lines. Intensity ratios of the test and flow controls will be calculated to determine positivity.

Furthermore, by employing the use of a cytokine based assay, positively-diagnosed patients can regularly monitor cytokine levels providing crucial insight to clinicians of disease progression and to identify appropriate treatment regimen especially with the currently strained and limited allocation of health resources.

By using reporters with higher sensitivities, a variety of rapid diagnostics can be performed on small sample quantities allowing for little to no sample prep further reducing time to result and increasing field deployability.

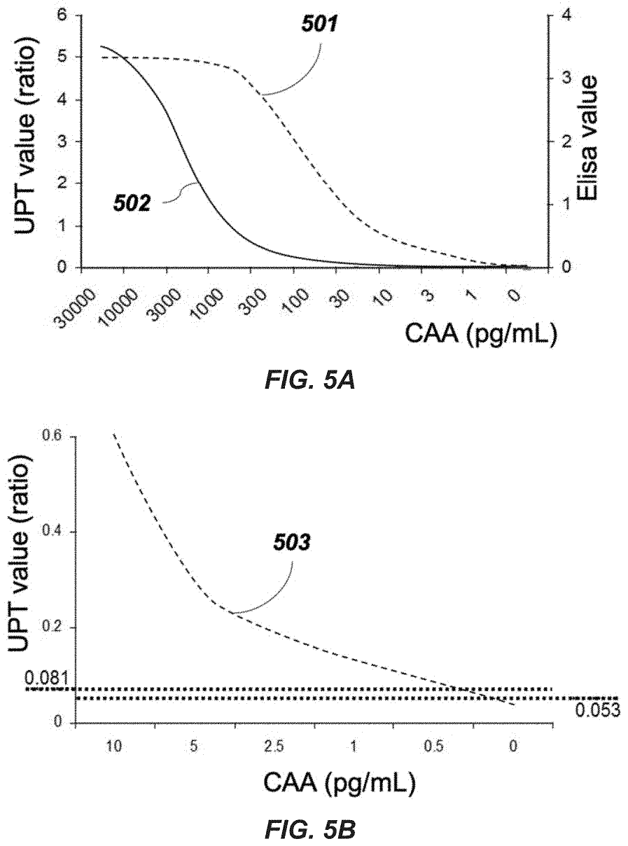

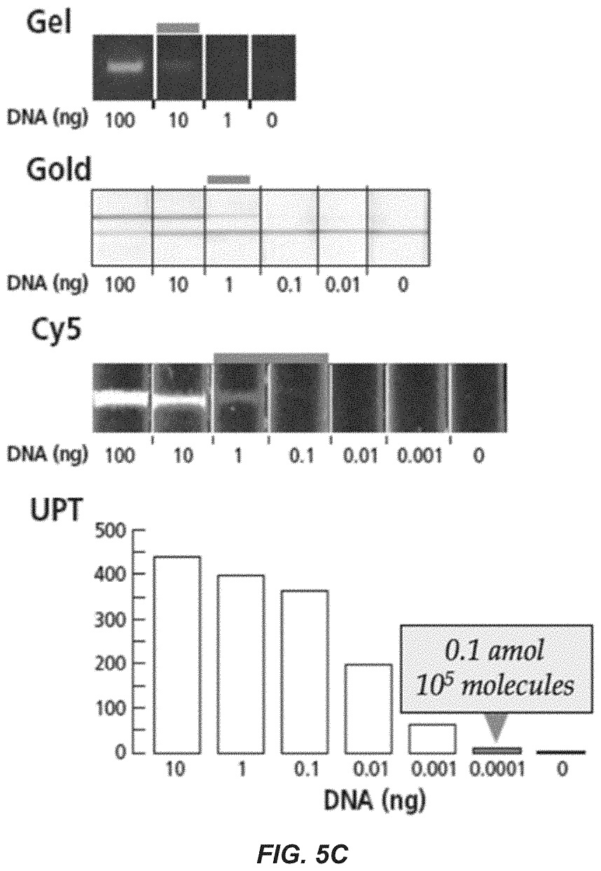

The disclosed rapid diagnostic platform has shown superior sensitivity compared to both gold and ELISA based assays as shown in FIGS. 5A-5C. FIGS. 5A and 5B depict the Analytical Sensitivity (FIG. 5A) and Limits of Detection (FIG. 5B) of the disclosed platform (“UPT”) (501, 503) compared to ELISA (502) in a urine-based CAA assay for schistosoma. In this assay, the test line capture molecule is PGL-1 antibody and the control line capture molecule is Rabbit anti-Goat IgG. The nanoparticle conjugate utilized Goat anti-Human IgM conjugated to the rare earth nanoparticle. The LOD for this assay reached sub-picogram/ml levels. The data presented in FIG. 5C shows results from a comparison of various assay formats (gel, gold, Cy5 vs. disclosed platform) against the disclosed platform for the detection of digoxigenin from V. chlorae. The results show significant increase in sensitivity compared to gold with the disclosed optical reporters achieving sub-attomolar limits of detection.

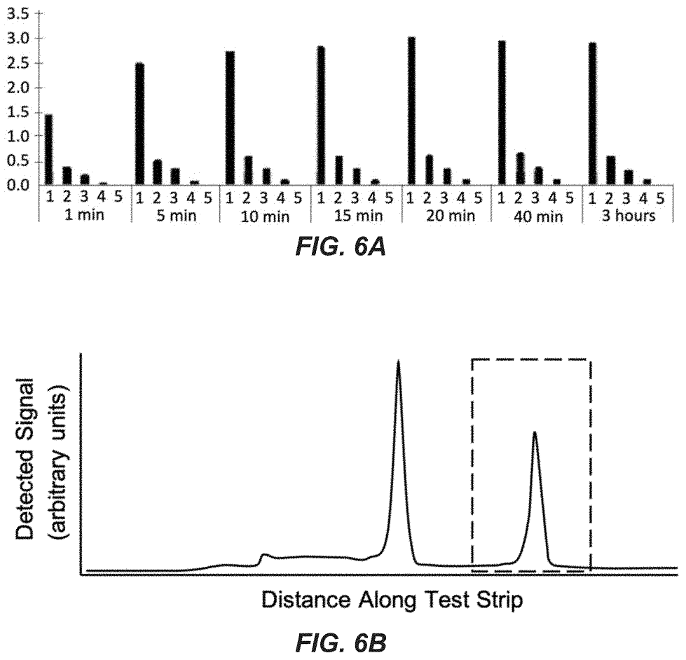

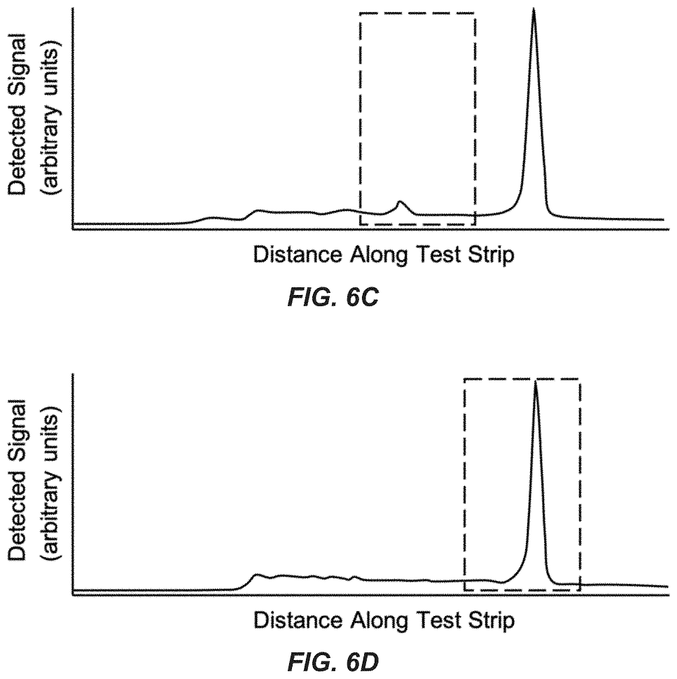

Another significant advantage enabled by the increased sensitivity and limits of detection of the disclosed optical reporters is the ability to achieve actionable results in less than 5 minutes. The low background noise and lack of autofluorescence of the rare-earth particle reporters provide the earliest detection possible. The data presented in FIGS. 6A-6C, is from a clinical trial for leprosy evaluating a rapid LF antibody-based assay using 5 different PGL-I specific, IgM antibodies. FIGS. 6A-6B show the time to result; within 5 minutes the Test and Flow Control lines stabilize providing an initial response, while FIG. 6C shows direct screenshots from the UC reader identifying Positive, Borderline positive and Negative patients. This work was performed as part of the PEOPLE project (Post ExpOsure Prophylaxis for LEprosy, in the Comoros and Madagascar).

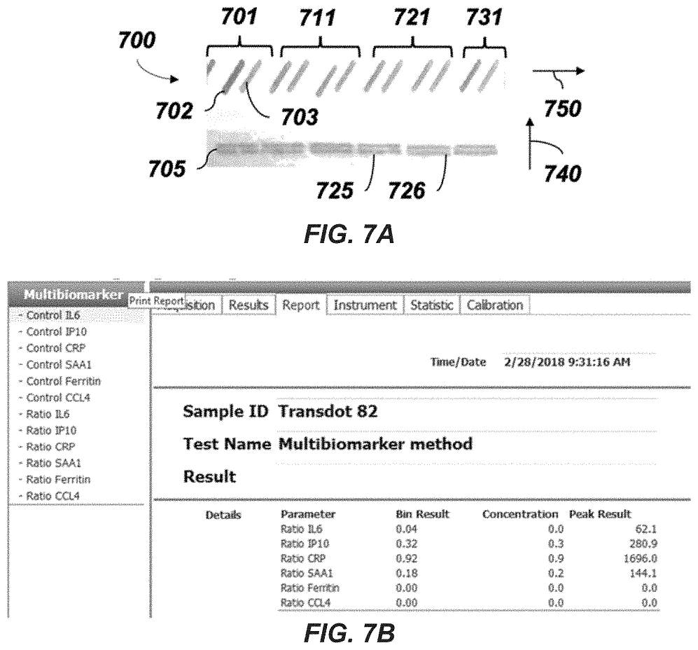

FIGS. 7A and 7B detail a multi-biomarker test for select cytokines to identify active Tuberculosis infections. The ratios of different cytokines provide actionable insight into the presence of an infection but also enables the ability to follow the disease progression. FIG. 7A shows a portion of an alternative test strip arrangement. In particular, the test strip (700) is configured to test for multiple analytes using groups of test and control lines. In this example, Group 1 (701) is testing for CRP only shows a single full pair of test lines (702) and control lines (703). Group 2 (711) shows two full pairs of test and control lines for IP-10, Group 3 (721) shows two full pairs of test and control lines for IL-6, and Group 4 (731) shows a single full pair of test and control lines for TNF-alpha. Other cytokines were tested; this image only shows a portion of the test strip. As can be see, each pair of test and control lines has a corresponding sample pad (705, 725, 726). When the sample is applied, the sample flows in the flow direction (740), past the test and control lines. The test strip is then scanned in the scan direction (750). In preferred embodiments, it is scanned multiple times, at different distances from the sample pads in the flow direction.

FIG. 7B provides an example display output in a disclosed system, based on the test strip described by FIG. 7A.

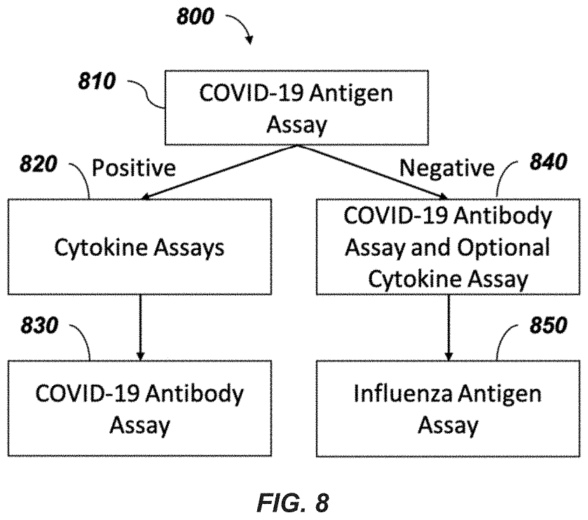

An embodiment of a typical testing methodology using the disclosed system and method for COVID-19 is outlined in FIG. 8. In this embodiment of a method (800), a subject would be administered a test. The test strip would comprise at least a COVID-19 Antigen Assay (810), and preferably would also contain a COVID-19 Antibody assay, a multiplexed cytokine assay, and an influenza assay, although the others may be provided on other test strips that can be administered at the appropriate time if desired. In the event of a positive result from the antigen assay, it is useful to examine the multiplexed cytokine assay (820) to establish current cytokine baselines and to monitor progression of disease processes (i.e., cytokine cascade/respiratory inflammation). Additionally, the ability to monitor IgM/IgG/IgA ratios in known positive individuals has shown clinical utility in guiding the treatment plan, so an antibody assay (830) may optionally be used for that purpose. In the instance of a negative antigen based test (810), a COVID-19 Antibody Assay (840), and optionally a multiplexed cytokine assay, can be examined to determine if the individual has been exposed to COVID-19 in the past. In addition, optionally examining the results from an influenza antigen assay will allow the assessment of whether the subject has been infected with, e.g., an influenza virus.

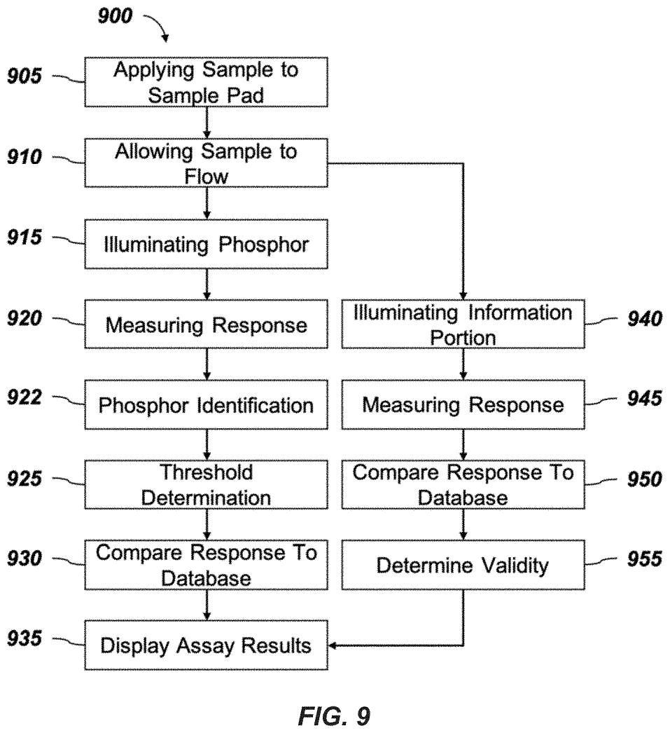

FIG. 9 provides a general flowchart for a method for rapidly and securely diagnosing an infection. As seen, the method (900) generally requires that at least a portion of a test sample from a patient be applied (905) to a sample pad on a disclosed test strip, as described above. The sample is allowed to flow across the test strip (the sample flowing over at least one test line on the test strip) for a predetermined period of time. After that point in time, a laser, LED, or other appropriate radiation source illuminates rare-earth particles on one or more test lines on the test strip (915). The illumination uses at least one wavelength of light that at least some of the rare-earth particles are capable of absorbing. In preferred embodiments, this illumination step involves illuminating at least one test line that comprises two or more different rare-earth particles, each conjugated to a different analyte.

Once the rare-earth particles have absorbed that radiation, they will emit light. That responsive light can be detected by a sensor and measured (920). For example, the sensor could generate a signal based on the received light, and passes that signal to a processor. The processor then translates the signal into an intensity at a given point in time, and builds an emission profile based on the received signals from the sensor (and therefore based on the emissions of rare-earth particles on the test line). In other examples, the sensor simply captures images of the test line, which captures the emissions from the individual rare-earth particles. A processor can then use image processing techniques to identify the position and intensity of light that was captured, and can mathematically calculate the total intensity of the phosphors captured by the test line.

The processor may then optionally identify one or more of the rare-earth particles, based on the measured response. For example, variables related to the emission profile of each rare-earth particle, such as the position of the rare-earth particles, the rise time, the decay time, and/or the peak wavelength of the captured emissions, etc., can be used to determine what rare-earth particles are being detected and measured. This may be done by comparing a variable related to the emission profile of the response to a value in a database to determine what analyte is being assayed. For example, if each rare-earth particle on a test strip has a different decay time, the decay time can be measured and compared to a database correlating the decay times to a particular rare-earth particle and/or a particular analyte.

The processor can then optionally determine if the response (i.e., the intensity) is greater than a predetermined threshold (925) for that assay. This threshold can, e.g., be used to determine if the response is in a previously validated test range. For example, at a very low intensities (i.e., very low concentrations of rare-earth particles emitting), the sensor readings might not have been validated as being accurate or reproducible, even if the sensor is detecting a particular rare-earth particle appears to be present on a test line. Alternatively, a calibration curve may not be valid if the intensity is below a particular threshold. This threshold can also be helpful for discerning between definitive diagnoses and possible diagnoses. That is, if the intensity is less than a first threshold, the result will be considered “negative”, if the intensity is between a first and second threshold, the result will be considered “possible”, and if the intensity is greater than the second threshold, the result will be considered “positive”.

The response can then be compared to a database (930). The database may, e.g., contain a calibration curve that correlates intensity emitted by a certain rare-earth particle type with the concentration of the analyte in the test sample. As discussed previously, the database may contain information correlating the rare-earth particle emission profile with a specific type of rare-earth particle and/or a specific test assay. If the rare-earth particle and/or analyte has not already been identified, that identification may occur here as well.

Once this information is in hand, the assay results can be displayed (935). Preferably, the processors in the system automatically identifies the rare-earth particles detected, and the associated analyte. In some embodiments, the emitted response curve is provided. In some embodiments, a “positive/negative” or “positive/possible/negative” approach is use to display the results. In some embodiments, the measured intensity is displayed. Preferably, the analyte is displayed along with the associated result.

The method (900) may optionally include an additional couple of steps that can be used to provide additional information or security relating to the test strip. For example, at some point in the process, a separate portion of the test strip that contains phosphors may be illuminated (940), a portion that is not a test line or a control line. This portion provides information about the test strip. The rare-earth particles absorb the radiation and emit a response, which is detected and measured (945) (generally, it is detected by a sensor and the measurement is performed by a processor, although other configurations are possible).

This response is compared to a database (950) to determine information about the test strip. For example, the rare-earth particles could be selected and printed in particular positions such that the emission profile encodes information that describes, for example, a manufacturing date, an expiry date, a lot code, and/or a code representing the types of analytes that are being tested.

Once this information is in hand, the processors can determine if the assay is valid (955), based on the determined information about the test strip. For example, processor determines that the current date is beyond the expiry date of the test strip, the processor could cause a warning to be displayed, or a warning noise to occur, alerting a user of a problem and indicating the test may not be valid. Or, the processor could compare a list of expected analytes to a list of analytes that were identified based on the rare-earth particles present in the test lines, to ensure they match, and providing a warning or error message that the test may not be valid if they do not.

It should be noted that FIG. 9 shows these additional steps as occurring almost in parallel with steps 920-930, but they can occur at any time, and may be combined, in part, with one or more of the steps previously discussed. For example, it is clear that a comparison of the expiry date with a current date could occur prior to the sample being applied to the sample pad—thus steps 940-955 could occur prior to step 905.

Those skilled in the art will recognize, or be able to ascertain using no more than routine experimentation, many equivalents to the specific embodiments of the invention described herein. Such equivalents are intended to be encompassed by the following claims.

What is claimed is:

1. A rapid diagnostic test strip, comprising:
a substrate;
a sample pad on a proximal portion of the substrate;
a conjugate release pad located distally from the sample pad, the conjugate release pad comprising at least two conjugating materials, each conjugating material capable of conjugating to an analyte, and each conjugating material comprising a rare earth particle, each rare earth particle having a single pure crystalline phase of a rare earth-containing lattice, a uniform three-dimensional size, and a uniform polyhedral morphology;
an absorbent pad located distally from the conjugate release pad; and
at least one test line adapted to bind to at least one of the analytes.

2. The rapid diagnostic test strip according to claim 1, wherein each analyte is one or more of coronavirus antigen variants, an influenza antigen, IgM, IgG, IgA, or one or more cytokines.

3. The rapid diagnostic test strip according to claim 1, wherein the at least one test line comprises a single test line capable of binding to two or more analytes.

4. The rapid diagnostic test strip according to claim 1, wherein the conjugate release pad further comprises a control material, comprising at least one rare-earth particle that is different from other rare-earth particles used for the two or more conjugating materials.

5. The rapid diagnostic test strip according to claim 1, where at least one of the two or more conjugating materials is configured to absorb at least one different wavelength from at least one other rare-earth particle in the conjugate pad.

6. The rapid diagnostic test strip according to claim 1, where in at least one of the two or more conjugating materials is configured to emit at least one different wavelength from at least one other rare-earth particle in the conjugate pad.

7. The rapid diagnostic test strip according to claim 1, further comprising a control line distantly located from the at least one test line.

8. The rapid diagnostic test strip according to claim 1, wherein the one or more test lines are on a top surface of a diagnostic pad, which is positioned between the conjugate pad and the absorbent pad.

9. The rapid diagnostic test strip according to claim 1, further comprising one or more rare-earth particles attached to the top surface of the diagnostic pad, where a variable relating to the emission profiles of the one or more rare-earth particles provide information about the test strip.

10. The rapid diagnostic test strip according to claim 9, wherein the variable is temporal.

11. The rapid diagnostic test strip according to claim 9, wherein the information about the test strip comprises which analytes the test strip is configured to assay, a date, or a combination thereof.

12. A rapid diagnostic system, comprising:
at least one rapid diagnostic test strip according to claim 1; and
a reader adapted to detect the presence of a rare-earth particle bound to a test line.

13. The rapid diagnostic system according to claim 12, wherein the reader contains a processor configured to:
cause the reader to emit at least one wavelength of light that a rare-earth particle on a test strip is capable of absorbing;
allow the reader to detect an emission profile of the rare-earth particle in response to the at least one wavelength of light after at least one wavelength of light is no longer being emitted;
determine if a parameter of the responsive emission profile is above a predetermined threshold;
determine a temporal response of the rare-earth particle based on the detected emission profile; and
provide a test result based on the temporal response.

14. The rapid diagnostic system according to claim 12, wherein the at least one rapid diagnostic test strip comprises a first test strip configured to assay for an influenza virus and an assay for a coronavirus.

15. The rapid diagnostic system according to claim 14, wherein the first test strip is further configured to assay for IgM, IgG, IgA, at least one cytokine, or a combination thereof.

16. The rapid diagnostic system according to claim 14, wherein the at least one rapid diagnostic test strip comprises:
a singleplex Multi-Antibody Assay (IgM, IgG, IgA);
a multiplex Antibody Assay (IgM, IgG, IgA);
a singleplex Cytokine Assay (Cytokine Panel);
a multiplex Cytokine Assay (Cytokine Panel);
a multiplex Antibody/Cytokine Assay (IgM, IgG, IgA/Cytokine Panel); or
a combination thereof.

17. A method for rapidly and securely diagnosing an infection, comprising:
applying at least a portion of a test sample from a patient to a sample pad on a rapid diagnostic test strip according to claim 1;
allowing the sample to flow across the rapid diagnostic test strip for a predetermined period of time;
illuminating rare-earth particles on at least one test line with at least one wavelength of light the rare-earth particles are responsive to;
measuring a first response, the first response being an emission of light from the rare-earth particle; and
making a determination as to whether the first response is above a predetermined threshold.

18. The method according to claim 17, wherein illuminating the at least one test line comprises illuminating at least one test line comprising two or more different rare-earth particles, each rare-earth particle conjugated to a different analyte.

19. The method according to claim 17, further comprising comparing the first response to a value in a database to determine what analyte is being assayed;

20. The method according to claim 17, wherein at least one rare-earth particle attached to the test strip outside of the at least one test line is also illuminated, and the method further comprises:
measuring a second response to the illumination of the at least one rare-earth particle attached to the test strip outside of the at least one test line; and
comparing the second response to a database to determine information about the test strip;
determining if the assay is valid based on the determined information about the test strip; and
displaying the results of the assay.
